(19) United States
(12) Patent Application Publication
(10) Pub. No.: US 2010/0168233 A1
(43) Pub. Date: Jul. 1, 2010

(54) TERBINAFINE FORMULATION

(75) Inventors: Dalia Jayes, Modiin (IL); Boaz Nitzan, Sde-varburg (IL); Michal Royz, Rehovot (IL); David Barak, Modiin (IL); Orit Sholto, Beit-gamliel (IL); Rachel Mosckovitz-Silversmith, Rishon Le Zion (IL); Shirly Ben-Yaakov Duady, Ramat Gan (IL); Doron Firedman, Karmei Yosef (IL)

Correspondence Address: BANNER & WITCOFF, LTD., 1100 13th STREET, N.W., SUITE 1200, WASHINGTON, DC, 20005-4051, US

(73) Assignee: POWER PAPER LTD, Petah Tikva (IL)

(21) Appl. No.: 12/528,023

(22) PCT Filed: Feb. 20, 2008
(86) PCT No.: PCT/IL2008/000218

§ 371 Date: Feb. 17, 2010

Related U.S. Application Data
(60) Provisional application No. 60/902,346, filed on Feb. 21, 2007.

(51) Int. Cl. A61K 31/191; A61K 31/137; A61P 31/10

(52) U.S. Cl. 514/557; 514/655

(57) ABSTRACT

The present invention provides a pharmaceutical composition comprising as the active agent a terbinafine compound, water, and at least one water-soluble or water-miscible nonionic surfactant, wherein the terbinafine compound has at least one form selected from the group consisting of free base form, acid addition salt form, ionic form, and combinations thereof; and wherein substantially no alcohol is present.

### FIELD

The present invention relates to a terbinafine anti-fungal composition. Moreover, the present invention is of a terbinafine anti-fungal composition formulated for delivery by iontophoresis.

### BACKGROUND

Terbinafine, a synthetic allylamine is commonly used to treat fungal infection. Terbinafine inhibits ergosterol synthesis by inhibiting squalene epoxidase to result in destruction of the fungal cell wall.

The art discloses oral and topical formulations of terbinafine hydrochloride, such as LAMISIL, which is marketed by Novartis. Oral administration of terbinafine hydrochloride may be used in the treatment of onychomycosis, however this route of administration is associated with undesirable side effects such as hepatotoxicity. Available topical formulations of terbinafine base or terbinafine hydrochloride are not effective in the treatment of onychomycosis.

A known method for delivering some active agents into the skin is iontophoresis. Iontophoresis is a method of electrical delivery of a substance.

A substance to be delivered by iontophoresis should preferably be charged in order to respond to an electric current. In cases wherein the substance is not naturally charged, the substance can be combined with a charging agent or subjected to environmental conditions such as a specific pH environment, which induces charge formation. Properties of a substance or composition such as, but not limited to size of the active molecules, pH, viscosity, hydrophobicity, hydrophilicity and competitive ions all affect iontophoretic delivery of a substance. Furthermore, the physical state of the composition needs to be configured for practicality and ease of use with iontophoresis.

The available terbinafine compositions are formulated for oral or topical administration and have not been formulated for delivery using iontophoresis.

It would therefore be advantageous to have a terbinafine formulation, which has been formulated for effective delivery by iontophoresis, such as is provided by the present invention.

### SUMMARY

Aspects of the invention include anti-fungal formulations comprising terbinafine. In one aspect the anti-fungal terbinafine formulation may be configured for delivery by iontophoresis. The formulation may comprise a terbinafine compound in at least one or a combination of free base form, acid addition salt form and ionic form, water, and at least one water-soluble or water-miscible nonionic surfactant, wherein substantially no alcohol is present.

Another aspect relates to a formulation of terbinafine and acetic acid or a salt of terbinafine and acetic acid.

A further aspect relates to a device, such as an iontophoretic device, which may include, or be used with, the anti-fungal terbinafine formulation configured for delivery by iontophoresis.

An additional aspect relates to the use of the device and the terbinafine formulation for treatment of a fungal infection.

### BRIEF DESCRIPTION OF THE DRAWINGS

The various features of the invention will best be appreciated by simultaneous reference to the description which follows and the accompanying drawings and in which:

FIG. 1a shows schematically a device for iontophoretic delivery of a terbinafine formulation according to one aspect of the present invention;

FIG. 1b shows schematically a device attached to a toe according to one aspect of the present invention;

FIG. 2 shows schematically a method of treatment according to one aspect of the present invention;

FIG. 3 illustrates graphically the effect of increased current density on the delivery of terbinafine into the receiving compartment;

FIG. 4 shows a graphical representation of the effect of increased current density on the delivery of terbinafine into nails;

FIG. 5 shows a graphical representation of the effect of increased current density on the delivery of terbinafine into the receiving compartment, following diffusion for 120 hours;

FIG. 6 shows a graphical representation of the influence of NaCl concentrations on terbinafine delivery from a formulation containing 2.5% terbinafine HCl into the receiving compartment of the passive and active groups;

FIG. 7 shows a graphical representation of the influence of NaCl concentrations on terbinafine delivery from a formulation including 2.5% terbinafine HCl into the nails of the passive and active groups;

FIG. 8 shows a graphical representation of the influence of terbinafine concentration on its delivery into the receiving compartment; and

FIG. 9 shows a graphical representation of the influence of terbinafine concentration on its delivery into the nail.

### DETAILED DESCRIPTION

It was discovered that iontophoretic delivery of terbinafine is effective in the treatment of onychomycosis and does not result in the systemic side effects caused by the oral administration route. In the context of the present application, the term “iontophoresis” means any method of electrical delivery of substances, including electrotransportation, iontophoresis, electroosmosis, electroporation, and/or a combination thereof.

The Formulation/Composition

Aspects of the invention relate to a composition of terbinafine. In some aspects the composition includes at least one active agent, at least one solvent, and at least one surfactant.

The active agent is N,6,6-trimethyl-N-(naphthalen-1-ylmethyl) hept-2-en-4-yn-1-amine (terbinafine) of structural formula:

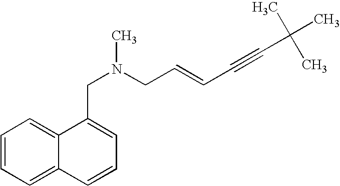

or any suitable derivative thereof. In some aspects the active agent terbinafine is in at least one of free base form or in acid salt form or in ionic form or a combination thereof. The active agent terbinafine may be in cis form, trans form and any combination thereof or in a racemic form.

Terbinafine base and terbinafine HCl exhibit different chemical properties, such as water solubility, hygroscopicity, stability and lipophilicity. Terbinafine base is significantly less water soluble than the HCl salt. Terbinafine base is hygroscopic, which may cause stability problems in a formulation. As such the requirements for a formulation including the base as compared to the HCl salt may be different. Delivery of terbinafine using iontophoresis from a formulation containing the terbinafine base compared to delivery from a formulation containing terbinafine HCl may exhibit differences. It further appears that the antifungal activity of the terbinafine base and salt against different fungi are not the same. Therefore, in some applications it may be desirable to have a formulation comprising the terbinafine base which is substantially free of terbinafine HCl in order to optimize activity or a formulation comprising the combination of the terbinafine base and salt.

In an aspect, wherein the composition is for delivery by iontophoresis, the composition is formulated to facilitate at least one or a combination of (a) a charged active terbinafine, (b) solubilized terbinafine, (c) a non-clustered terbinafine, (d) suitable molecular dispersion, (e) stability and (f) a physical state which is conducive for movement of the terbinafine active ions to a treatment site under the influence of current.

In some aspects the conductivity properties of the formulation may be tailored for iontophoresis. In some aspects the conductivity properties of the formulation may be tailored for iontophoretic delivery into the nail, wherein the formulation properties and conductivity properties may be different for delivery into the nail than for delivery into the skin. In some aspects a conductive medium, such as an aqueous solution of an active substance may be used in the formulation. In some aspects wherein excipients are included, the excipients may be non ionic excipients in order to reduce or prevent delivery of competition ions instead of active terbinafine ions. In an alternative aspect, monovalent ions of non-actives may be included in the formulation in order to create a higher ion flux to push the anti-fungal active into the nail. In some aspects the pH is modified to modify the degree of ionization of the terbinafine.

In one aspect the terbinafine composition comprises an effective amount of ionized terbinafine. The terms “suitable percentage or effective amount of ionized terbinafine” as used herein refers to an amount of ions which would be at least adequate for delivery by current into the nail. In an alternative aspect, the synthesized composition may be further processed during patient treatment to result in ionized terbinafine. One non-limiting example of a process which may result in ionizing the terbinafine is lowering of the pH of the composition during delivery of the active drug by an iontophoresis device, as a result of for example electrolysis products. Such a process may be referred to as ‘in-situ ionization’. The amount of ionized compound is dependent on the pH of the composition. Typically, the pH of the composition should be lower than the pKa of the compound for facilitating a composition comprising greater than 50% of ionized compound. In an aspect wherein the terbinafine composition of the present invention is for delivery by iontophoresis, the pH of the composition may be lower than the pKa of terbinafine, which is about 7.1. As such, the pH of the terbinafine composition may be formulated at a pH of about 7.1 or below or may be formulated at a higher pH which is lowered during treatment. In one aspect the pH of the composition before application of the composition to an iontophoresis device and a body area is greater than about 4.7 and the pH during application is less than about 4.7. In a further aspect, the composition may be formulated at a pH of above about 4 and either lowered during treatment or maintained at this value.

In an aspect, wherein the formulation is for topical use, the parameters of the formulation may be controlled to afford an uncharged terbinafine formulation. Due to the positive charge of the keratin in the nail, a charged terbinafine formulation may be undesirable in topical treatment.

In some aspects the composition may further comprise a suitable pH modifier. Non-limiting examples of a suitable pH modifier include triethanolamine, sodium hydroxide, acetic acid, lactic acid and sodium acetate. The amount of pH modifier to be added may be calculated in order to achieve a suitable pH which results in a suitable amount of ionized drug. Care should also be taken that the pH is not too low, such that it would result in damage to the area of the body to be treated. As such the pH may be optimized according to the parameters of therapeutic acceptable values and optimal ionization of the active drug.

In some aspects, the composition may further comprise a buffer. A buffer may maintain the pH of the formulation at a certain level. In one aspect a buffer system of acetic acid and sodium acetate is used to maintain the pH of the formulation between about pH 3 and 4.5. Additional non-limiting examples of suitable buffers include citrate/citric acid, citric acid/sodium hydrogen phosphate and sodium acetate/acetic acid.

The active agent is present in any suitable amount. A suitable amount may be an amount which will provide optimal therapeutic activity, but which will not result in toxicity. In some aspects, the amount is determined in order to deliver an amount of terbinafine which is above the minimum inhibitory concentration (MIC). The minimum inhibitory concentration of terbinafine hydrochloride for dermatophytes is about 0.0015 μg/ml. The proportion of terbinafine used in the composition of the present invention may range from about 0.05% to about 15% w/w. In some aspects the percentage of terbinafine in the composition is from about 0.25% to about 4% w/w. In some aspects, terbinafine is present from about 0.1% to about 2% w/w or from about 0.5% w/w to about 1% w/w. In one aspect the terbinafine is present in about 1% w/w. In a further aspect, terbinafine is present in about 0.5% w/w.

In one aspect the composition comprises at least one solvent. The solvent may function to solubilize the active compound and/or to facilitate an ionized state of the active agent terbinafine. In one aspect the solvent is water of any suitable purity, such as but not limited to double deionized water. In an aspect, wherein the active agent is terbinafine, terbinafine is hydrophobic and as such the solubility of terbinafine in water is low.

In some aspects an additional suitable solvent may be included. The additional solvent may aid in solubilizing the terbinafine, in for example water. One criteria for a suitable solvent is the ease that the solvent solubilizes the terbinafine. Additional considerations in the choice of solvent may include, but are not limited to not causing sensitivity, to stability and to oxidative stability to heat and current and to non-volatility. As such, even though terbinafine is very soluble in methanol and ethanol, in one aspect, the composition of the present invention does not include substantially any alcohol. Evaporation of alcohol due to external conditions may result in a change of the proportion of the formulation ingredients. Evaporation of alcohol may also result in terbinafine precipitation. In some aspects, the formulation of the present invention may be used in hot environments or in combination with heating of the affected body area, conditions which would promote evaporation of an alcohol. It has been found by the inventors that the percentage of terbinafine base or terbinafine salt in the formulation influences the distribution of the active drug in the nail. Under certain conditions, it was observed that although with iontophoretic delivery of a composition comprising more than 2% terbinafine more terbinafine may be delivered to the nail than with delivery of a 1% terbinafine formulation, less terbinafine was delivered to the nail bed with the formulation containing the greater amount of terbinafine. Very volatile solvents in a formulation may evaporate under storage and treatment conditions and raise the percentage of active terbinafine in the formulation preventing optimal treatment of the nail bed. In a case such as in onychomycosis wherein the active drug target delivery site is the nail bed, the use of an alcohol containing formulation may not be desirable. The present invention provides in one aspect, a pharmaceutical composition comprising a terbinafine compound in at least one or a combination of free base form, acid addition salt form and ionic form, water, and at least one water-soluble or water-miscible surfactant, wherein substantially no alcohol is present. In a further aspect, the present invention provides a terbinafine formulation substantially free of any ingredient, with a volatility comparable to ethanol, such that the ingredient has a boiling point comparable to ethanol and which is therefore substantially volatile at room temperature.

In one aspect the composition includes at least one water-soluble or water miscible surfactant. The surfactant may be non-ionic. In an aspect wherein the surfactant is non-ionic, the surfactant may substantially not produce ions which could compete with the active terbinafine ions for delivery by iontophoresis. The surfactant may exhibit at least one or a combination of functions, which include emulsifying the terbinafine, aiding in solubilizing the low water-soluble terbinafine in the solvent such as water, facilitating dispersion of the terbinafine, stabilizing the terbinafine and facilitating terbinafine which can move under current. In some aspects the surfactant may also facilitate gelling of the composition. One non limiting example of a suitable water-soluble non-ionic surfactant is an amphiphilic polymer, such as polyoxyethylene-polyoxypropylene co-polymers, for example poloxamers. Poloxamer may also facilitate gelling of the composition. Alternative non-limiting examples of a suitable surfactant include 2-(2-Ethoxyethoxy)ethanol (ethoxydiglycol), polyglyceryl-10 oleate; nonoxynol-9, oleth-20, decyl gluceth-20, Dimethicone Copolyol, Steareth-20, Ceteareth-20, Steareth-21, Isoceteh-20, Oleth-20, Oleth-10, Laureth-23, Nonoxynol-10, PEG-40 hydrogenated castor oil, PEG-35 castor oil, PEG-7 glyceryl cocoate; Tween 80, Span 80, decaglyceryl dipalmitate and combinations thereof. In some aspects, the surfactant may have anti-fungal properties, which may result in combination or synergistic anti fungal activity with the terbinafine. The surfactant may be present in any suitable amount, such as but not limited to from about 5% to about 50% w/w.

In some aspects, the formulation may include at least one preservative in any suitable amount. The preservative may be present in an amount of less than about 2% w/w. The preservative may be water soluble. Non-limiting examples of suitable preservatives include methylisothiazolinone, Sharonmix MTG, phenoxyethanol, methylparaben and derivatives thereof.

In some aspects, the formulation may include at least one penetration enhancer in any suitable amount. At least one penetration enhancer may be present in an amount of less than about 20%. A penetration enhancer may aid in delivering the active drug into the skin and/or nail. Non-limiting examples of penetration enhancers include urea, acetic acid, salicylic acid, dimethyl sulfoxide, ethoxydiglycol, Isoceteh-20, dimethyl isosorbide and combinations thereof. Surprisingly, the inventors have observed that in some formulations, inclusion of urea in the formulation may inhibit delivery of terbinafine to the nail bed. Any suitable penetration enhancer as known in the art may be used.

In some aspects, the formulation may include at least one conductivity enhancer, such as but not limited to NaCl, KCl, sodium sulfate, sodium citrate, sodium iodide, sodium acetate, potassium acetate, sodium lactate, potassium phosphate and combinations thereof in any suitable amount. The conductivity enhancer may be present in an amount up to about 5%. In one aspect the conductivity enhancer may be present in an amount up to about 1%. It has been observed by the inventors that a concentration of greater than about 2% of conductivity enhancer may inhibit iontophoretic drug delivery of terbinafine from the formulation of the present invention, which may be due to competition.

In some aspects the formulation of the present invention is formulated to have a conductivity above about 1.0 mSi/cm. In one aspect, the formulation of the present invention at a pH of about 4.5 has a conductivity range from about 3.0 to about 25.0 mSi/cm.

In some aspects, the formulation may include stabilizers, such as but not limited to cellulose derivatives, PVA, PVP, MC and HPMC.

In some aspects, the formulation may include at least one additional active agent, such as anti-fungal agents, antibiotics, anti-virals, analgesics and combinations thereof. In one aspect, at least one non-terbinafine constituent of the formulation may also have anti-fungal properties.

In some aspects, the formulation may include spore activators for activating fungal spores. In some aspects, electrical stimulation of the device is configured to activate spores, which may then be treated with the composition of the present invention.

In some aspects the formulation may include additional excipients known in the art of pharmaceuticals and cosmetics, such as but not limited to a colorant, thickeners, anti-oxidants, emulsifiers, humectants, and perfume.

The composition of the present invention may be formulated in any suitable physical form, such as, but not limited to a liquid, gel, cream, fluid, spray, dispersion or emulsion. In some aspects, the formulation which comprises terbinafine, water and a non-ionic surfactant, is in a gel form. The gel form is conducive for facile handling and facile use with an iontophoresis device. In some aspects, as described hereinabove the non-ionic surfactant is the gelling agent. Alternatively, a gelling agent may be added to the formulation in addition to the non-ionic surfactant. Any suitable gelling agent may be used, such as but not limited to hydroxyethylcellulose, hydroxymethylcellulose, methylcellulose, xanthan gum, guar gum, hydroxypropylcellulose and combinations thereof.

In one aspect, the formulation of the present invention may be combined in any suitable way with a hydrogel, including ionic and non-ionic hydrogels. In some aspects a suitable hydrogel may include sulfonic groups, and/or carboxylic and/or quaternary ammonium groups.

In a further aspect, the present invention provides a salt of terbinafine with acetic acid, such as terbinafine acetate or any suitable salt formed from a reaction product of terbinafine with acetic acid. The present invention provides a pharmaceutical composition comprising a salt of terbinafine with acetic acid. The composition may further include at least one solvent. In one aspect, the Composition comprises terbinafine acetate, water, and at least one water-soluble or water-miscible nonionic surfactant. The composition may further include terbinafine base and acetic acid. The composition may include any suitable excipient as described hereinabove. The terbinafine acetic acid salt and/or formulation thereof may be combined in any suitable way with any suitable hydrogel.

Further, the present invention provides any suitable combination of acetic acid with terbinafine, such as with the terbinafine base and/or with terbinafine HCl. and/or a terbinafine acid salt and/or with ionized terbinafine. In one aspect the formulation may be made from only acetic acid, water and terbinafine. In an alternative aspect, the formulation may further include a hydrogel. Optionally, a fragrance or a means for neutralizing the characteristic smell of acetic acid may be included in the formulation.

The acetic acid terbinafine salt may be used in the preparation of a medication for the treatment of any suitable disorder, such as the treatment of a fungal infection. In one aspect, the acetic acid terbinafine salt may be for treatment of onychomycosis. The inventors have shown using in vitro testing that iontophoretic delivery of a formulation including terbinafine acetate resulted in delivery of about 150 μg/cm² of terbinafine to the nail bed. Under similar conditions, a formulation of terbinafine base resulted in delivery of about 10 μg/cm² to the nail bed and a formulation of terbinafine HCl resulted in delivery of about 45 μg/cm² to the nail bed. The terbinafine acetic acid formulation resulted in significantly greater delivery of terbinafine to the nail bed than the alternative forms of terbinafine.

The acetic acid salt may be prepared by any suitable method. In one aspect, the salt is prepared by reacting terbinafine free base with acetic acid. The acetic acid may be added to the terbinafine free base. Any suitable amount and concentration of acetic acid may be used. In one aspect up to about 99% w/w acetic acid was used. In one example 95% acetic acid is mixed with terbinafine base. The product may then be isolated.

Alternatively, the acetic acid salt may be made in situ in a formulation which may include acetic acid, terbinafine base and additional ingredients, by reaction of acetic acid with terbinafine base and wherein the terbinafine acetate is not isolated.

A further method includes in situ generation of the acetic acid. A formulation may be prepared which includes terbinafine base and an acetate containing compound, such as for example an acetate salt, for example sodium acetate or acetic anhydride. Current is applied to the formulation by for example application of an iontophoresis device and the current facilitates electrogeneration of acetic acid, by for example release of a proton during electrolysis. The generated acetic acid may then react in situ with the terbinafine free base to form terbinafine acetate. This method is not limited to preparation of the acetic acid salt, but may be used to make any suitable acid salt of terbinafine.

The terbinafine acetate and/or mixture of terbinafine and acetic acid may be added to a hydrogel. It was observed in in vitro studies that hydrogel acetic acid terbinafine formulations in which there were higher concentrations of terbinafine delivered more terbinafine to the nail bed than a similar formulation with a lower concentration of terbinafine. For example a hydrogel acetic acid terbinafine formulation with about 2.67% w/w terbinafine delivered substantially less terbinafine than a formulation including about 5.5% w/w terbinafine.

### The Device

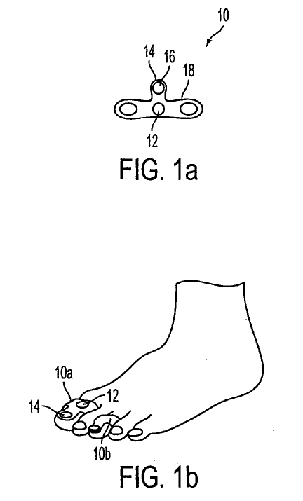

In one aspect the present invention provides a device for treatment of onychomycosis comprising an iontophoresis device, wherein the iontophoresis device comprises a terbinafine composition of the present invention. In an aspect, wherein the terbinafine formulation is for delivery by iontophoresis, the formulation may be used in combination with any suitable iontophoresis device. The terms “device,” and “iontophoresis device,” as used herein include “iontophoretic patch,” “electrically operated device,” and “electrically operated patch,” and will interchangeably stand for any method or device, used for electrical delivery of substances, including electrotransportation, iontophoresis, electroosmosis, and electroporation. The device may be any device of the art, which may be thin and flexible or non-thin and/or non-flexible. The term thin as used herein refers to less than about 5 mm thick. The term ‘flexible’ as used herein refers to the device being foldable and/or conformable to any body surface, such as, but not limited to a toe or finger. The device may be a light weight device. In one aspect the device may have a weight of from about 2 g to about 10 g. In some aspects the device may weigh more or less. The device may be powered by any suitable power source, such as, but not limited to a galvanic couple or a battery which may be integral to the device or may be an external component. The device may be manufactured to include the terbinafine formulation of the present invention or alternatively, the formulation may be applied separately, such as before use, for example as part of a kit. In one aspect, a kit for treatment of onychomycosis features an iontophoresis device and a terbinafine composition of the present invention. Non-limiting examples of suitable devices are described in US patent application, Publication No. 20050038375 A1 which is incorporated by reference herein. FIG. 1a shows one non-limiting example of an iontophoresis device 10 which is suitable for use with the formulations of the present invention and which is described in US patent application, Publication No. 20050038375 A1. Device 10 includes a counter electrode 12, an active electrode 14 and a power source 16 disposed on a frame 18, wherein the counter electrode 12 and active electrode 14 are electrically connected to the power source 16. FIG. 1b shows the device 10a or 10b attached to a toe.

In an aspect, wherein the formulation which comprises terbinafine, water and a surfactant, is in a gel form, the gel may be disposed on the device or on the treatment area of a body, without problems of leaking. The gel may be disposed directly or indirectly on any suitable element of the iontophoresis device, such as on at least one electrode, or may be disposed on/in a holding element, such as a non-woven formulation retainer or on/in a hydrogel. A formulation comprising less than about 10 mg of terbinafine may be applied to the device. In one aspect, a formulation comprising less than about 2 mg terbinafine may be applied to the device. In one aspect, a formulation comprising up to 1 mg of terbinafine may be applied to the device for treatment.

The terbinafine ions are positively charged and as such the formulation may be disposed in contact with the anode/s, such that the current resulting from the iontophoresis device may promote delivery of the charged terbinafine ions from the anode/s into the affected body area, such as the skin and/or nail.

In an alternative aspect wherein alternating current is used the terbinafine formulation may be disposed under both the anode and cathode.

The device may be configured to provide any suitable current density to deliver the terbinafine formulation to the nail. In an aspect, wherein the device is for delivering terbinafine specifically to the nail bed and nail matrix, a current density of greater than about 100 μA/cm² may be provided. In an aspect wherein the formulation includes up to about 1% terbinafine and the device is for delivering terbinafine to the nail bed and nail matrix a current density of about 400 μA/cm² and higher may be provided. It was found by the inventors that increasing the current density increased the delivery of terbinafine into the nail, however the difference in amount of terbinafine delivered between the lower current density and the higher current density was not pronounced. Surprisingly, a pronounced difference was observed in the distribution of the terbinafine using higher current density compared to the lower current density. At a current density of above 300 μA/cm² significantly more terbinafine was delivered from the formulation to the nail bed.

### Topical Use

The terbinafine formulation of the present invention may be used for topical treatment of a fungal infection. In an aspect, wherein the terbinafine formulation is for topical use, the formulation as described hereinabove, which may be a gel may be applied to an affected body area, such as the nail and/or skin to treat the area. The formulation may be applied directly to the body or alternatively may be included in a passive patch, which may then be applied to the affected body area. Any suitable passive patch as described in the art may be used.

### Treatment of Fungal Infection

In one aspect the present invention provides a method of treating a fungal infection comprising administering a therapeutically effective amount of a terbinafine composition of the present invention. The terms ‘treatment’ ‘treat’ and ‘treating’ as used herein encompass any treatment of a fungal infection, such as onychomycosis and includes: preventing the infection or disease from occurring in a subject which may be predisposed to the disease; inhibiting the infection or disease, i.e. arresting its development; and/or relieving the disease, i.e. causing regression of the disease. Relieving the disease means attaining improvement in the subject's condition, including, but not limited to clinical improvement, microbiological improvement and aesthetic improvement.

The terbinafine formulation of the present invention may be used in the treatment of any suitable fungal infection. In some aspects, the terbinafine formulation is for use in the treatment of a fungal infection caused by at least one of dermatophytes, candida and molds and combinations thereof. In one aspect, the terbinafine formulation of the present invention is for treating onychomycosis. Onychomycosis is a fungal infection of the nails and surrounding skin. In the most common form of onychomycosis, the fungus invades the nail bed under the nail plate, beginning at the hyponychium and then migrating proximally through the underlying nail matrix. Typically, oral treatment of onychomycosis delivers the active drug to the nail bed in order to treat the infection.

The fungal infection, such as onychomycosis may be treated by applying an iontophoresis device to the infected nail area. The device may include the terbinafine formulation, such as described hereinabove in a suitable physical state, such as a gel or fluid state. The terbinafine formulation may optionally be contained in a retainer or other drug holding means. In an aspect, wherein the formulation is not attached to the device, the formulation may be applied to the electrode/s of the device or applied directly to the nail region to be treated.

In one aspect, the device may include a means such as a membrane, which is permeable to the terbinafine ions, and substantially impermeable to at least one other constituent of the formulation. Such a membrane may be configured so that only the active terbinafine molecules or ions will contact the nail. Other formulation ingredients, which may be competitive with the ions or may be deleterious to the skin or nail may be prevented from contacting the nail or skin. Non-limiting examples of a suitable selective barrier membrane include an ion-exchange membrane and/or a specific pore size membrane.

The terbinafine composition may be administered by iontophoresis. The subject may contact the infected nail area to be treated with the device. In some aspects, the contact of the device with the body area causes closing of the circuit of the device with the nail and the current promotes delivery of the terbinafine ions from the formulation onto and into the nail and surrounding areas for treatment of the nail.

The device may be removed from the body area at the end of the device application time. Time of application can vary. In some aspects, application time is from about 1 hour to about 24 hours or equivalent thereof. Equivalent time of application, means that the time of application can be divided up, with the total time being the same. For example the device and/or formulation may be applied for a time of for example 24 hours, which is equivalent to application for eight hours on each of three days. In some aspects, application time is from about 5 hours to about 24 hours. In one aspect, application time is overnight, such that the composition may be administered overnight. However, in some aspects application time may be less or more. The device may be removed from contact with the body area after a time period, which can optionally be predetermined or is determined according to the desired dosage, the time it takes for the electrode to be depleted, or until sufficient effect or no more improvement can be seen. After removal of the device, the active drug may be further delivered by for example diffusion from the nail plate and upper layers of the nail to the nail matrix and nail bed. The treatment regimen and the frequency of treatment may be designed to take this into account. For example, treatment of the nail may be repeated until saturation or near saturation of the nail with the active drug, after which a time period is waited until the active drug or a proportion of the active drug is distributed to the nail bed and/or nail matrix and/or until the nail is no longer saturated or near saturated. The waiting period after drug saturation may be several days or even several weeks. The waiting period may be at least about 3 days or more. Saturation may be defined as when substantially no more or a very reduced amount of drug is being delivered into the nail from the formulation as compared to initial delivery.

In some aspects a pretreatment can be applied prior to use of the device. Non-limiting examples of pretreatments include applying a cleanser, applying a moisturizing composition, cutting nail, removing dry skin, bathing, softening treatment, applying an anti-irritant, applying a permeation enhancer, heating, microporation, electrical stimulation, applying a formulation comprising a pharmaceutically active ingredient, applying a formulation comprising a cosmetically active ingredient or a combination thereof.

In one aspect a pretreatment of urea and a salt, such as NaCl is applied to the infected nail or surrounding area or a combination thereof. The pretreatment may include urea from about 10% to about 30% w/w and NaCl from about 1% to about 10% w/w. The infected area may be pretreated for any suitable time which may range from about 20 minutes to about 2 hours. In some embodiments pretreatment may be up to abut 24 hours. A means for occluding the pretreatment site may be used. A suitable means for occlusion may include a plaster. The pretreatment may be applied without or with current.

In some aspects a post treatment can be applied to the body area after application of the device. Non-limiting examples of post treatments include applying an occlusion formulation, applying a cleanser, cooling, applying a nail varnish, applying a formulation comprising a pharmaceutically active ingredient, topical application of a terbinafine formulation of the present invention, applying a formulation comprising a cosmetically active ingredient or a combination thereof.

The treatment can optionally be a one-time treatment or can be repeated in suitable time intervals any suitable number of times. In one aspect, the treatment is once a week or is repeated daily or several times a week for a period of about a month or up to about 3 months. In some aspects, treatment may be for more than 3 months, for example up to about a year. In one aspect, treatment may be daily to achieve saturation of the nail with terbinafine. Saturation or near saturation of the nail in some cases may take from about a week to about three weeks of daily treatment. Saturation may occur is some individuals after less treatment or after more treatment. A waiting period may be waited before additional treatment. The waiting period may be for a period of several weeks. In some cases the waiting period may be more or less. After the waiting period, additional booster treatments may be administered once or several times a week, which may be consecutive or non-consecutive treatments. The booster treatments may be administered for a period of up to about one year. Use of the present invention can facilitate alleviation and elimination of the fungal infection. Duration of effect can be affected by time and frequency of application, type and amount of current used, severity of condition and inactivation of the terbinafine delivered and present in the nail and matrix. The duration of the effect of the treatment may vary. Repeated use may have a synergistic effect on duration and extent of treatment result.

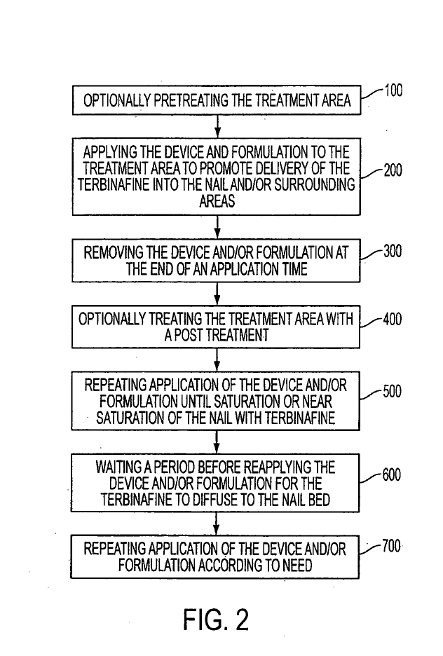

FIG. 2 shows schematically a typical treatment according to the present invention. The treatment area may be pretreated (100) as described hereinabove. The device and formulation may then be applied to the treatment area of the nail and/or surrounding skin area such that a current promotes delivery of terbinafine to the nail and/or surrounding area (200). The device and/or formulation may be removed from the treatment area after a time as defined hereinabove (300). The treatment area may be optionally treated with a post treatment as described hereinabove (400). The same device, a different device or a different sample of the same device may again be applied and the treatment may be repeated at a suitable time interval as described hereinabove (500). The application may be repeated until saturation or near saturation of the drug in the nail. An average number of treatments for reaching saturation may be precalculated and the same number used for each patient or the number may be calculated according to each individual. A period may be waited in order for the drug to diffuse to the nail matrix and/or nail bed (600). An average waiting time for the drug to diffuse to the nail bed may be precalculated and used generally for each patient or the waiting time may be calculated according to each individual or certain parameters of an individual. Parameters may include, but are not limited to age, sex, severity of disorder, nail resistance, weight, height, and medical history. The treatment and the application of the device and/or formulation may be repeated according to need (700).

In one aspect, the treatment may be configured for home use. In other aspects, the treatment may be conducted in a supervised environment.

It is noted that oral administration of lamisil typically includes one 250 mg tablet taken daily. The formulation of the present invention is configured to deliver daily substantially less than 100 mg of terbinafine to the body. The formulation of the present invention can deliver daily substantially less than 1 mg of terbinafine to the body. In one aspect, the formulation of the present invention is configured to deliver to the nail bed more than a thousand times the MIC of terbinafine.

### Nail Penetration of Terbinafine

The following experiments 1-10 were performed to verify that the terbinafine formulation of the present invention facilitates delivery of terbinafine into the nail by passive delivery and by the influence of current. The experiments were also designed to verify the improved delivery into the nail of terbinafine from the formulation of the present invention using current.

### Experiment 1

### In Vitro Penetration Study of Terbinafine from a Terbinafine Formulation According to an Aspect of the Present Invention Using Active Iontophoresis

An in-vitro penetration study was conducted to verify penetration into the nail of terbinafine from a formulation of the present invention using Franz cells. A formulation comprising terbinafine HCl (1%), Pluronic 127 (20%) and double deionized water (79.0%) was used. It was found using porcine nails that under the influence of current 100 μA/cm² using a powered iontophoresis device with graphite delivery electrode and silver/silver chloride counter electrode, that terbinafine was delivered into the nail in an amount which was above the MIC.

### Experiment 2

### In Vitro Penetration Study of Terbinafine from a Terbinafine Formulation According to an Aspect of the Present Invention Using Active Iontophoresis

An in-vitro penetration study was conducted to verify penetration into the nail of terbinafine from a formulation of the present invention using Franz cells. A formulation comprising terbinafine HCl (1%), Pluronic 127 (25%), Methylparaben (0.3%), Sharonmix MTG (0.1%) and double deionized water (73.6%) was used. It was found using porcine nails that under the influence of current 100 μA/cm² using a powered iontophoresis device with graphite delivery electrode and silver/silver chloride counter electrode, that terbinafine was delivered into the nail in an amount which was above the MIC.

### Experiment 3

### In Vitro Penetration Study of a Terbinafine Formulation According To an Aspect of the Present Invention Using Passive Delivery (Topical Application)

An in-vitro penetration study was conducted to verify penetration into the nail by passive delivery of terbinafine from a formulation of the present invention using Franz cells. A formulation comprising terbinafine HCl (1%), Pluronic 127 (25%), Methylparaben (0.3%), Sharonmix MTG (0.1%), and double deionized water (73.6%) was used. It was found using porcine nails that terbinafine was delivered into the nail in an amount which was above the MIC.

### Experiment 4

### Comparison of In-Vitro Penetration of Terbinafine from a Terbinafine Formulation of the Present Invention Using Topical Delivery Compared to Active Iontophoresis

In-vitro delivery of terbinafine into porcine nails was measured from a formulation comprising terbinafine HCl (1%), Pluronic 127 (25%), Methylparaben (0.3%), Sharonmix MTG (0.1%) and double deionized water (73.6%) either passively with no current or under the influence of current 100 μA/cm² using a powered iontophoresis device with graphite delivery electrode and silver/silver chloride counter electrode. It was observed that the amount of terbinafine delivered into the nail using current (active iontophoresis) was over one and a half times the amount of terbinafine which was delivered into the nail using passive delivery.

### Experiment 5

### Determination of Delivery of Terbinafine from the Terbinafine Formulations of the Present Invention into the Nail Bed

Terbinafine formulations were prepared as detailed herein. Porcine hooves were used as a model to screen formulations. The tissue was removed from porcine legs within a few hours of sacrifice, and hooves were stored frozen for a period of no longer than 12 months. In vitro delivery studies were performed using Franz diffusion cells with Neoflon nail adapters specially designed to hold the nails. Several nails were used in each test (active) or control (passive) group.

Prior to each delivery experimental nails were immersed in double distilled water (DDW) for 24 hours to allow complete hydration. Each nail was mounted in the adapter with the outer dorsal surface open to drug formulation and the inner ventral surface in contact with phosphate buffer saline (PBS) solution (5 ml) in the receiving compartment. The mounted nails were incubated at 37° C. during the experiment. Aliquots of 500 μl formulation were applied to the exposed dosing area of the nail, and then sealed to avoid evaporation.

In each active cell, the Ag/AgCl cathode electrode was inserted into the receiver chamber and the Graphite electrode was fixed in the donor chamber. A power supply was used for the application of a constant direct current. Drug formulation was placed in the donor compartment. Samples of 0.1 ml were drawn from the receiving compartment at specific intervals and the amount of terbinafine was measured by HPLC. Passive experiments (without current application) were also performed for comparison with active experiments. At the end of each experiment the formulation was removed from the donor compartment. The PBS solution was collected from the receiving compartment and nails were collected for HPLC analysis. Nails were cleaned and the dosing area was cut into small pieces and incubated with DDW for an average of 16 hours. Prior to HPLC analysis terbinafine was eluted from the nails.

Terbinafine formulation was applied to the surface of the nail only once, for 24 hours. Then, the formulation was removed from the donor compartment and the cells remained assembled for an additional 120 hours without an electric current. A sample (100 μl) was taken from the receiving compartment (time=0). An additional sample was taken from the receiving compartment at 48 hours from time 0. At the end of the experiment, (120 hours from time 0), cells were disassembled and the nails and PBS solution from the receiving compartment (nail bed) were collected for HPLC analysis. The amount of terbinafine in nails and in the receiving compartment buffer was measured by HPLC.

In an alternative experiment terbinafine formulation was applied to the surface of the nail once daily 24 hours apart, for a total of 72 hours. Following incubation for 24 hours, the formulation was removed from the donor compartment and the nail was cleaned. Subsequently, a “fresh” formulation was added to the donor compartment for an additional 24 hours. Following nail incubation with the formulation for 48 hours, a sample (100 μl) was taken from the receiving compartment (nail bed). Then, after 72 hours of incubation, the nail, the formulation and the PBS taken from the receiving compartment were collected for HPLC analysis.

### Experiment 6

The effect of increasing current densities on terbinafine penetration (1% terbinafine formulated with 1% NaCl w/w) into the nail plate and into the nail bed (the receiving compartment) was examined using the method described in experiment 5. Increased current densities of 100 to 500 μA/cm² were applied and sampling of the receiving compartment was performed after 48 and 72 hours. Nail sampling was performed after 72 hours. The control experiment included the delivery of the same formulation using passive diffusion cells (without electrical current).

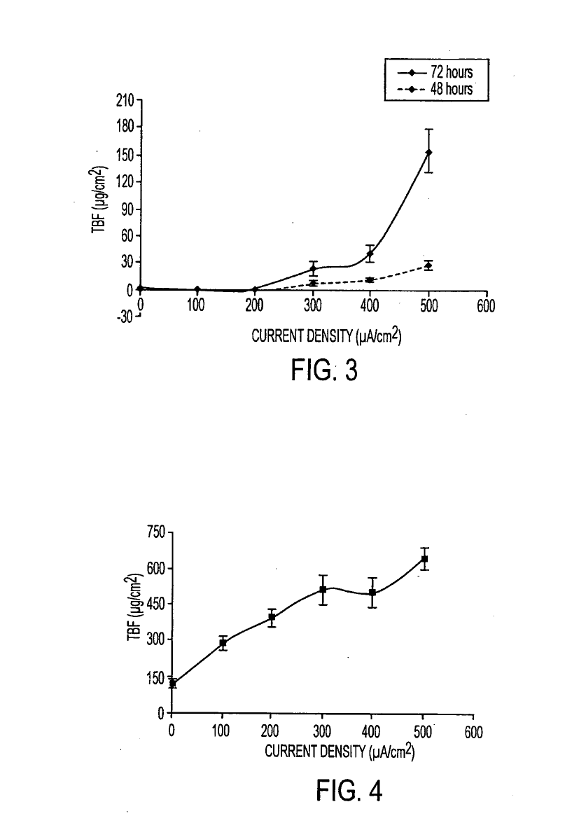

In the receiving compartment (nail bed), a significant increase in the terbinafine content was recorded between the active groups (400 and 500 μA/cm²) compared to the passive group, suggesting that the drug penetrates into the deeper layers of the nail and into the nail bed only at high current densities as shown in FIG. 3.

In the nails, a significant increase in the terbinafine content was recorded between each active group (100, 200, 300, 400 and 500 μA/cm²) compared to the passive group as shown in FIG. 4.

### Experiment 7

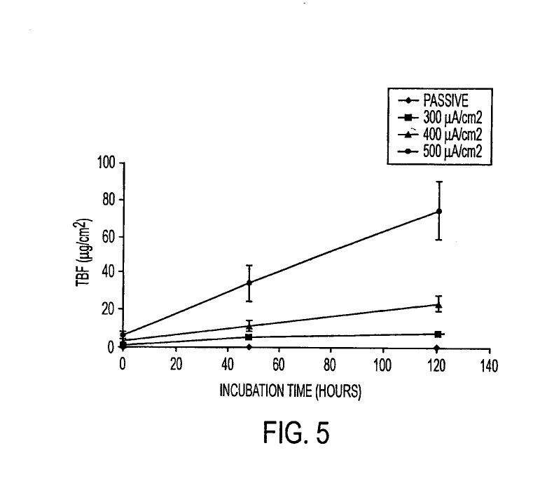

The diffusion of terbinafine from the nail into the nail bed was examined after a single dose of the drug. Nails were incubated with the formulation containing 1% terbinafine HCl and 1% NaCl w/w for 24 hours under current densities of 300, 400 or 500 μA/cm². The control experiment included the delivery of the same formulation using passive (without electrical current) diffusion cells. As described above in experiment 5, the formulation was removed from the donor compartment after 24 hours and cells remained assembled for an additional 120 hours without an electric current. The terbinafine content (μg/cm²) in the nails and the receiving compartment (nail bed equivalent) is shown in FIG. 5. As can be seen, only a low amount of terbinafine, was detected in the receiving compartment of all tested groups immediately after 24 hours of incubation with the formulation. Following diffusion for two additional periods of 48 and 120 hours, an increase in the drug content was recorded only at higher current densities of 400 and 500 μA/cm². In the passive control cells, no Terbinafine was detected in the receiving compartment at any of the time points.

### Experiment 8

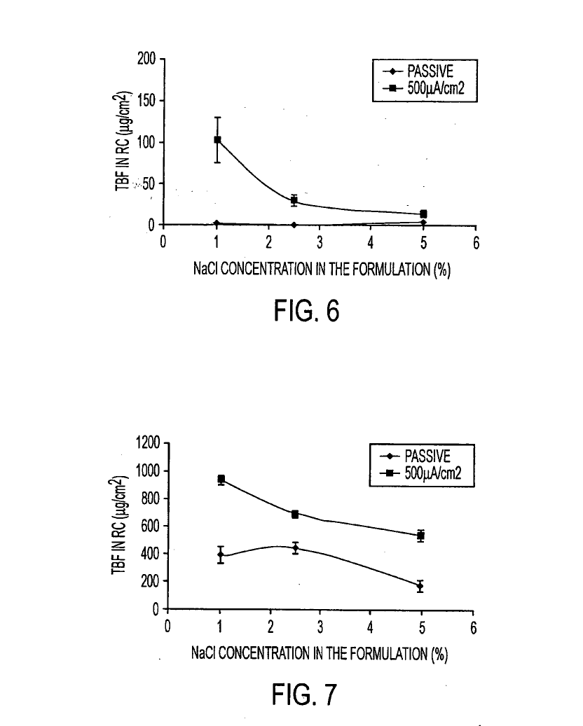

The effect of increased NaCl concentrations on the delivery of 1% or 2.5% terbinafine HCl was tested. A single current density of 500 μA/cm² was applied for 72 hours. As a control, a passive group was tested under the same conditions without electric current. Increasing NaCl concentrations from 1% to 2.5 or 5% in a formulation of 1% terbinafine HCl did not increase the drug delivery into the nails and the receiving compartments, in the active or in the passive groups. In the formulation containing 2.5% terbinafine HCl, increasing NaCl concentrations from 1% to 2.5 or 5% significantly inhibited the drug delivery into the nails and the receiving compartments in the active group as shown in FIGS. 6 and 7.

### Experiment 9

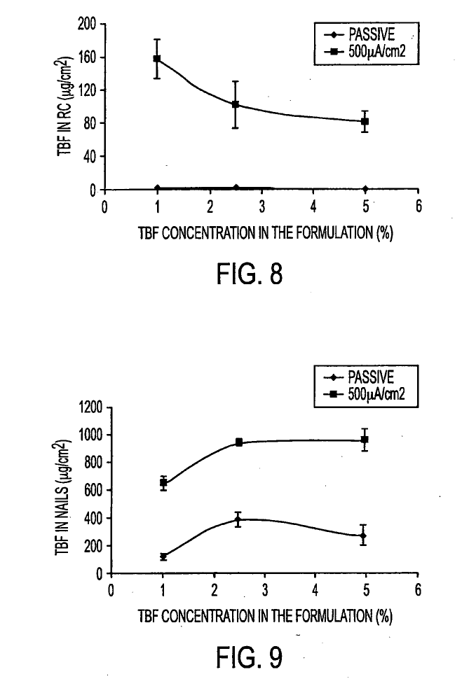

The effect of drug concentration on iontophoretic delivery of terbinafine HCl through porcine nails was tested. Different formulations were prepared containing different concentrations of terbinafine HCl (1%, 2.5% or 5%) and 1% NaCl. In these experiments, a single current density of 500 μA/cm² was applied for 72 hours. As a control, a passive group was tested under the same conditions without application of current. Increasing the concentration of terbinafine in the formulations from 1% to 2.5% or to 5% significantly increased terbinafine content in the nails of the active group as shown in FIG. 9. The increase in the passive group was mainly between 1% and 2.5%. In contrast with the increased content in the nails, terbinafine content significantly decreased in the receiving compartment (nail bed) of the active group. In the passive group, no difference was recorded in terbinafine content in the receiving compartment as shown in FIG. 8.

### Experiment 10

Urea was tested for its potential to enhance penetration of terbinafine HCl into both the nail plate and the nail bed. Porcine nails were incubated with a formulation containing 1% terbinafine, 1% NaCl and 20% urea for 72 hours. A control experiment was performed using the same formulation without urea. Both formulations were examined under a current density of 500 μA/cm². The presence of 20% urea in the formulation significantly decreased the content of the drug in the receiving compartment compared to the control group. There was no difference in the drug content in the nail between the two groups with or without urea.

Reference is now made to the following examples, which together with the above descriptions, illustrate the invention in a non limiting fashion.

### Example 1

The following formulation was prepared:

| Component | % w/w |
|---|---|
| deoinized water | 73.6 |
| Plutonic F127 | 25 |
| Terbinafine HCl | 1 |
| Methylparaben | 0.3 |
| Sharonmix MTG | 0.1 |

The pH of the formulation was 2.98 and the conductivity was 1315 microsiemens/cm.

### Example 2

The following formulation was prepared:

| Component | % w/w |
|---|---|
| deoinized water | 74.1 |
| Plutonic F127 | 25 |
| Terbinafine HCl | 0.5 |
| Methylparaben | 0.3 |
| Sharonmix MTG | 0.1 |

Methylparaben was dissolved in water with heating. The mixture was stirred and then the aqueous mixture was cooled to between 0° C. about 4° C. Pluronic 127 and Sharonmix MTG were then added and stirred until the emulsifier was completely dissolved. Terbinafine HCl was then added to the clear liquid solution and the mixture stirred with cooling until complete dissolution. The resulting composition had a pH of between about 3.0 to about 4.0. Terbinafine is positively charged at this pH range.

### Example 3

The following formulation was prepared as described above for Example 2:

| Component | % w/w |
|---|---|
| deoinized water | 78.6 |
| Pluronic F127 | 20 |
| Terbinafine HCl | 1 |
| Methylparaben | 0.3 |
| Sharonmix MTG | 0.1 |

### Example 4

The following formulation was prepared as described above for Example 2:

| Component | % w/w |
|---|---|
| deoinized water | 68.6 |
| Pluronic F127 | 30 |
| Terbinafine HCl | 1 |
| Methylparaben | 0.3 |
| Sharonmix MTG | 0.1 |

### Example 5

The following formulation was prepared as described above for Example 2:

| Component | % w/w |
|---|---|
| deoinized water | 79 |
| Pluronic F127 | 20 |
| Terbinafine HCl | 1 |

### Example 6

The following formulation was prepared as described above for Example 2:

| Component | % w/w |
|---|---|
| deoinized water | 83.6 |
| Pluronic F127 | 15.0 |
| Terbinafine HCl | 1 |
| Methylparaben | 0.3 |
| Sharomix MTG | 0.1 |

### Example 7

The following formulation was prepared.

| Component | % w/w |
|---|---|
| Double distilled water | 52.6 |
| Poloxamer 407 | 30.0 |
| Terbinafine base | 1 |
| Acetic acid/sodium acetate | 5.0 |
| KCl | 1.0 |
| Sharonmix MTG | 0.1 |
| Methylparaben | 0.3 |
| Nonoxynol-9 | 10.0 |

Methylparaben was dissolved in water with heating. The mixture was stirred and then the aqueous mixture was cooled to between 0° C. and about 4° C. Pluronic 127 and Sharonmix MTG were then added and stirred until the emulsifier was completely dissolved. Nonoxynol-9, Acetic acid and Sodium Acetate were then added and stirred until completely dissolved. Terbinafine base was then added to the clear liquid solution and the mixture stirred with cooling until complete dissolution. The resulting composition had a pH of between about 3.0 to about 4.0. Terbinafine is positively charged at this pH range.

### Example 8

The following formulation was prepared as described for Example 7.

| Component | % w/w |
|---|---|
| Double distilled water | 63.5 |
| Poloxamer 407 | 25.0 |
| Terbinafine base | 0.5% |
| Acetic acid/sodium acetate | 5.0 |
| KCl | 1.0 |
| Nonoxynol-9 | 5.0 |

### Example 9

The following formulation was prepared.

| Component | % w/w |
|---|---|
| Deionized water | 61.0 |
| 2-(2-Ethoxy)ethanol | 30.0 |
| Terbinafine base | 1.0 |
| KCl | 1.0 |
| Acetic acid/sodium acetate | 5.0 |
| hydroxyethylcellulose | 2.0 |

Terbinafine base was dissolved in a mixture of acetic acid and ethoxydiglycol.

This mixture was then dissolved in water with stirring. Sodium Acetate then was added and dissolved with stirring. The mixture was stirred with heating up to 40° C. Hydroxyethylcellulose was then added to the clear liquid solution and the mixture stirred with cooling until complete dissolution and gellification. The gel was stirred and was cooled to room temperature. The resulting composition had a pH of between about 3.0 to about 4.5. Terbinafine is positively charged at this pH range.

### Example 10

The following formulation was made.

| Component | % w/w |
|---|---|
| Double distilled water | Up to 100 |
| Methyl cellulose | 1.0 |
| Hydroxyethylcellulose | 4.0 |
| Terbinafine HCl | 1.0 |
| Glacial Acetic acid | 20.0 |
| KCl | 1.0 |
| Sodium hydroxide | To pH 3.5 |
| Polyglyceryl-10 Oleate | 10.0 |

Terbinafine HCl was dissolved in a mixture of acetic acid and Polyglyceryl-10 Oleate. This mixture was then dissolved in water with stirring. Sodium Hydroxide was subsequently added and dissolved with stirring. The mixture was stirred with heating to 40° C. Hydroxyethylcellulose and Methylcellulose were then added to the clear liquid solution and the mixture stirred with cooling until complete dissolution and gellification. The gel was stirred and was cooled to room temperature. The resulting composition had a pH of between about 3.0 to about 4.5. Terbinafine is positively charged at this pH range.

### Example 11

The following formulation was made as described in example 10.

| Component | % w/w |
|---|---|
| Double distilled water | Up to 100 |
| Hydroxyethylcellulose | 1.0 |
| Terbinafine HCl | 1.0 |
| Glacial Acetic acid | 20.0 |
| KCl | 1.0 |

The formulation was tested for delivery by iontophoresis and penetration to the nail bed using the method as detailed in Experiment 5. Following one time treatment, Terbinafine was found to be delivered to the nail bed in an amount greater than about 74 μg/cm².

### Example 12

The following formulation was made as described in example 9.

| Component | % w/w |
|---|---|
| Double distilled water | Up to 100 |
| Transcutol | 30.0 |
| Terbinafine base | 1.0 |
| Acetic acid | 5.0 |
| KCl | 1.0 |

The formulation was tested for delivery by iontophoresis and penetration to the nail bed using the method as detailed in Experiment 5. Terbinafine was found to be delivered to the nail bed in an amount greater than 30 μg/cm² after a one time treatment.

### Example 13

The formulation from example 9 was combined with hydrogel by dripping the formulation onto the hydrogel.

### Example 14

The formulation from example 10 was combined with hydrogel by dripping the formulation onto the hydrogel.

### Example 15

| Component | % w/w |
|---|---|
| Terbinafine base | 5% |
| Acetic acid | 95% |

Terbinafine base was mixed with acetic acid to form terbinafine acetate in solution.

### Example 16

The composition made in example 15 was combined with hydrogel by dripping onto the hydrogel so that the final concentration of Terbinafine base was 5.5%.

The present invention overcomes deficiencies of terbinafine compositions of the background art, wherein the terbinafine formulation of the present invention is configured for optimal delivery by iontophoresis, for improved delivery to the nail bed and for improved treatment of onychomycosis.

One skilled in the art can appreciate from the foregoing description that the broad techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

1-39. (canceled)

40. A pharmaceutical composition suitable for delivery by iontophoresis comprising a terbinafine compound in free base form, or acid addition salt form, or ionic form, or combination thereof, water, and at least one water-soluble or water-miscible nonionic surfactant.

41. The pharmaceutical composition of claim 40, wherein substantially no alcohol is present.

42. The pharmaceutical composition of claim 40, wherein the conductivity of the formulation is greater than about 1.0 mSi/cm.

43. The pharmaceutical composition of claim 40, wherein the surfactant is present in a concentration of greater than about 15% w/w and less than about 50% w/w.

44. The pharmaceutical composition of claim 40, comprising from about 0.1% terbinafine to about 2% terbinafine.

45. The pharmaceutical composition of claim 40, wherein the composition is a gel.

46. The pharmaceutical composition of claim 40, comprising an effective amount of terbinafine compound for topical treatment of a fungal infection.

47. The pharmaceutical composition of claim 40, for treatment of onychomycosis.

48. The pharmaceutical composition of claim 40, wherein the pH of the composition before application of the composition to an iontophoresis device and a body area is greater than 4 and the pH during application is less than 4.

49. The pharmaceutical composition of claim 40, further comprising a conductivity enhancer.

50. The pharmaceutical composition of claim 49, wherein the conductivity enhancer is present in an amount of less than 2% and the conductivity is greater than about 1.0 mSi/cm.

51. The pharmaceutical composition of claim 40, further comprising at least one of a gelling agent, a buffer, a pH modifier, a penetration enhancer, a preservative and a pharmaceutical excipient.

52. The pharmaceutical composition of claim 40, further comprising acetic acid.

53. The pharmaceutical composition of claim 40, where terbinafine comprises a salt of terbinafine with acetic acid.

54. The pharmaceutical composition of claim 40, comprising about 1% w/w terbinafine base and about 30% w/w 2-(2-Ethoxyethoxy)ethanol.

55. A device for treatment of onychomycosis comprising an iontophoresis device, wherein the iontophoresis device comprises the pharmaceutical composition of claim 40.

56. A method of treating a fungal infection comprising administering a therapeutically effective amount of the pharmaceutical composition of claim 40.

57. The method of claim 56 comprising:
treating a nail with a treatment wherein the treatment comprises:
applying an iontophoresis delivery device in contact with the nail, the surrounding area, or both;
contacting the composition of claim 1 with the iontophoresis delivery device for delivering terbinafine into the nail, surrounding area, or both;
removing the device at the end of a treatment time;
repeating the treatment a plurality of times until saturation or near saturation of the nail with terbinafine; and
allowing a waiting period after saturation of the nail before subsequent additional treatment.

58. A pharmaceutical composition for treatment of onychomycosis comprising terbinafine and terbinafine ions, water, and a conductivity enhancer, wherein the conductivity of the formulation is greater than 1.0 mSi/cm.

59. A pharmaceutical composition for delivery by iontophoresis comprising terbinafine base, terbinafine ions and water, wherein the conductivity of the formulation is greater than 1.0 mSi/cm.

60. A pharmaceutical composition comprising a terbinafine compound in free base form, or acid addition salt form, or ionic form, or combination thereof, water, and at least one water-soluble or water-miscible nonionic surfactant, wherein substantially no alcohol is present.
